(19) United States
(12) Patent Application Publication
(10) Pub. No.: US 2010/0087692 A1
(43) Pub. Date: Apr. 8, 2010

(54) HYDROGENATION METHOD AND PETROCHEMICAL PROCESS

(75) Inventors: Yuuji Yoshimura, Tsukuba-shi (JP); Makoto Toba, Tsukuba-shi (JP); Yasuo Miki, Tsuchiura-shi (JP); Masako Miki, Tsuchiura-shi (JP); Shigeru Hatanaka, Oita-shi (JP); Tetsuo Kudo, Oita-shi (JP); Tetsuo Nakajo, Yokohama-shi (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K., Minato-ku, Tokyo (JP)

(21) Appl. No.: 12/531,454

(22) PCT Filed: Apr. 14, 2008
(86) PCT No.: PCT/JP2008/057647

§ 371 Date: Sep. 15, 2009

(30) Foreign Application Priority Data
Apr. 19, 2007 (JP) 2007-110353

(51) Int. Cl. C07C 5/32; C07C 5/02

(52) U.S. Cl. 585/252; 585/265

(57) ABSTRACT

The present invention provides a hydrogenation method capable of converting cracked kerosene into the raw materials for petrochemical cracking having a high thermal decomposition yield by a hydrogenation reaction. The present invention is a petrochemical process for producing at least any of ethylene, propylene, butane, benzene or toluene by carrying out a thermal decomposition reaction at least using naphtha for the main raw material, wherein cracked kerosene produced from a thermal cracking furnace is hydrogenated using a Pd or Pt catalyst in a two-stage method consisting of a first stage (I), in which a hydrogenation reaction is carried out within the range of 50 to 180° C., and a second stage (II), in which a hydrogenation reaction is carried out within the range of 230 to 350° C., followed by re-supplying all or a portion of these hydrogenated hydrocarbons to a thermal cracking furnace.

### TECHNICAL FIELD

The present invention relates to a hydrogenation method for obtaining saturated hydrocarbons (hydrogenation) by adding hydrogen atoms to aromatic carbon-carbon double bonds and ethylenic carbon-carbon double bonds of a mixture of hydrocarbon compounds having aromatic ring and/or ethylenic carbon-carbon double bonds produced in the form of a fraction having a boiling point at 1 atmosphere (atm) of 90 to 230° C. (to be referred to as “cracked kerosene” or abbreviated as “CKR”) from a thermal cracking furnace in a petrochemical process for the production of ethylene, propylene, butane, benzene or toluene and the like by carrying out a thermal decomposition reaction using naphtha and the like as the main raw material (typically referred to as an ethylene production plant), and to a petrochemical process for re-using hydrocarbons hydrogenated by this method as raw materials for petrochemical cracker of thermal cracking furnaces.

The present application claims priority on Japanese Patent Application No. 2007-110353, filed on Apr. 19, 2007, the content of which is incorporated herein by reference.

### BACKGROUND ART

Ethylene plants produce such products as C4 fractions including ethylene, propylene, butane and butadiene, cracked gasoline (including benzene, toluene and xylene), cracked kerosene (C9 or larger fractions), cracked heavy oil (ethylene bottom oil) and hydrogen gas by thermal decomposition of naphtha and so on. In addition, each of the products produced by this thermal decomposition of naphtha are separated in a distillation process.

The following provides an explanation of a thermal decomposition process of naphtha in a typical ethylene plant, namely a process in which naphtha is converted to low molecular weight products containing olefins such as ethylene (25 to 30%) and propylene (15%) by thermal decomposition thereof.

In this process, the raw material naphtha passes through a large number of pipes in a thermal cracking furnace heated to 750 to 850° C. with a burner together with water vapor present for the purpose of dilution (weight ratio of 0.4 to 0.8 parts to 1 part raw material). Furthermore, the reaction pipes have a diameter of about 5 cm and length of about 20 m, and do not use a catalyst. Reactions including a decomposition reaction take place during the 0.3 to 0.6 seconds the naphtha passes through the high-temperature pipes. In addition, gas discharged from the thermal cracking furnace is immediately cooled rapidly to 400 to 600° C. to prevent further decomposition, and is further cooled by spraying recycled oil. Heavy components are separated from the cooled cracked gas in a gasoline rectifying tower. Water is then sprayed from above the tower in a subsequent quenching tower, and the water component and gasoline component (C5 to C9 components) are condensed and separated. Next, acidic gas (such as sulfur fractions and carbon dioxide gas) is removed in a soda washing tower (furthermore, hydrocarbons having 5 carbon atoms are described as C5 components, and this applies similarly to C9 components and so on). Hydrogen is separated by a low-temperature separator (−160° C., 37 atm) on the way. Methane, ethylene, ethane, propylene and propane are sequentially separated into pure components by passing through a distillation tower, respectively. This separation requires the use of a distillation tower having a large number of distillation plates of 30 to 100 plates each at a pressure of about 20 atm. Table 1 below shows a comparison of the components between ordinary naphtha and thermal decomposition products following thermal decomposition.

TABLE 1

| | Raw Material | Thermal Decomposition |
|---|---|---|
| | Naphtha (wt %) | Products (wt %) |
| H₂ | 0 | 0.75 |
| CH₄ | 0 | 15.0 |
| C₂H₄ | 0 | 26.5 |
| C₂H₆ | 0 | 5.2 |
| C3 component | 0 | 16.0 |
| C4 component | 2.0 | 8.5 |
| C5 or larger components | — | 28.1 |
| C5 component | 11.9 | — |
| C6 component | 16.4 | |
| C7 component | 17.8 | |
| C8 component | 27.5 | |
| C9 or larger components | 24.4 | |

These thermal decomposition products are mainly composed of a mixture of unsaturated hydrocarbon compounds having 9 or more carbon atoms, and the fraction having a boiling point at 1 atm of 90 to 230° C. is referred to as “cracked kerosene”. This cracked kerosene is a mixture of aromatic hydrocarbon compounds such as styrene, vinyltoluene, dicyclopentadiene, indane, indene, phenylbutadiene, methylindene, naphthalene, methylnaphthalene, biphenyl, fluorene or phenanthrene, aliphatic unsaturated hydrocarbon compounds and hydrocarbon compounds having both aromatic carbon-carbon double bonds and ethylenic carbon-carbon double bonds.

On the other hand, cracked kerosene has mainly only been used as products having low added value such as fuel, petroleum resin raw materials. Consequently, ethylene plants have been attempting to lower the ratio of these low added value products and increase the ratio of high added value products such as ethylene and propylene.

Among the low added value fractions produced from thermal cracking furnaces, saturated aliphatic hydrocarbon compounds such as ethane are re-supplied to the thermal cracking furnace where they are used as cracking raw materials, thereby making it possible to convert the ethane to ethylene and so on. On the other hand, even if cracked kerosene, itself, is re-supplied to the thermal cracking furnace and used as a cracking raw material, since many of the components thereof contain aromatic rings making them chemically stable, it is difficult to convert them to ethylene and other products having high added value by thermal decomposition.

In addition, these components also contain large amounts of easily polymerizable substances such as styrene having ethylenic carbon-carbon double bonds (in the form of vinyl groups and the like). Thus, in the case of supplying these substances to a high-temperature thermal cracking furnace directly, these substances undergo a thermal polymerization reaction, thereby resulting in the problem of the thermal cracking furnace easily being fouled by the resulting polymer (coke). Moreover, since these mixtures are composed of several tens of types of compounds, isolation of each component is unrealistic in economical terms.

Furthermore, an overview of the thermal decomposition process of naphtha is described in, for example, Organic Industrial Chemistry, Kagakudojin Co., Ltd., 11th edition, p. 58, “3. Production of Basic Synthesis Raw Materials by Decomposition (Cracking) of Naphtha”. In addition, a detailed description of the process flow of the thermal decomposition of naphtha is contained in Petrochemical Processes, Japan Petroleum Institute, ed., 1st edition, p. 21, “2. Olefins”.

The present invention relates to a reaction for hydrogenating cracked kerosene in two stages. Hydrogenation reactions of olefins and aromatic compounds along with catalysts used in those reactions are described in Japanese Unexamined Patent Application, First Publication No. H05-170671 and Japanese Unexamined Patent Application, First Publication No. H05-237391. More specifically, the Japanese Unexamined Patent Application, First Publication No. H05-170671 discloses a method for reducing the olefin content of raw material oils for hexane production by hydrogenation purification and activated clay treatment using Co/Mo, Co/Ni or Co/Ni/Mo and the like supported onto a carrier such as porous alumina or silica alumina. On the other hand, the Japanese Unexamined Patent Application, First Publication No. H05-237391 describes a method for forming diesel fuel having an improved cetane number by at least partially converting the aromatic substance to an acyclic substance together with saturating an olefin and an aromatic substance using a catalyst having palladium and platinum supported onto Y-type zeolite. Moreover, Japanese Patent No. 3463089 describes a hydrogenation catalyst preferable for use in the present invention.

### DISCLOSURE OF INVENTION

With the foregoing in view, an object of the present invention is to provide a hydrogenation method capable of converting cracked kerosene to raw materials for petrochemical cracker having a high thermal decomposition yield by a hydrogenation reaction, and to provide a petrochemical process by which useful components such as ethylene, propylene and cracked gasoline are obtained at high yield without easily causing fouling of the thermal cracking furnace by using such a hydrogenation method.

As a result of conducting extensive studies to solve the aforementioned problems, the inventors of the present invention found that cracked kerosene can be converted to raw materials for petrochemical cracker having a high thermal decomposition yield by a hydrogenation reaction by hydrogenating aromatic ring and/or ethylenic carbon-carbon double bonds present in the cracked kerosene in two stages consisting of stages (I) and (II) below followed by re-supplying to a thermal cracking furnace, thereby leading to completion of the present invention.

Namely, the present invention provides the means indicated below.

[1] A hydrogenation method comprising:

hydrogenating a mixture of hydrocarbon compounds having aromatic ring and/or ethylenic carbon-carbon double bonds in the following two stages (I) and (II):

(I) carrying out a hydrogenation reaction within the range of 50 to 180° C.; and

(II) carrying out a hydrogenation reaction within the range of 230 to 350° C.

[2] The hydrogenation method described in [1] above, wherein the mixture of hydrocarbon compounds having aromatic ring and/or ethylenic double bonds is a fraction consisting of hydrocarbons produced from a thermal cracking furnace using naphtha as the main raw material and having a boiling point within the range of 90 to 230° C. (referred to as “cracked kerosene”).

[3] The hydrogenation method described in [1] or [2] above, wherein a catalyst is used in the hydrogenation reaction, and the catalyst contains at least one type or two or more types of elements selected from the group consisting of palladium (Pd), platinum (Pt), ruthenium (Ru) and rhodium (Rh).

[4] The hydrogenation method described in [3] above, wherein the catalyst supplied to the hydrogenation reaction further contains at least one type or two or more types of elements selected from the group consisting of cerium (Ce), lanthanum (La), magnesium (Mg), calcium (Ca), strontium (Sr), ytterbium (Yb), gadolinium (Gd), terbium (Tb), dysprosium (Dy) and yttrium (Y).

[5] The hydrogenation method described in [3] or [4] above, wherein the catalyst supplied to the hydrogenation reaction is a catalyst supported onto zeolite.
[6] The hydrogenation method described in [5] above, wherein the zeolite is USY zeolite.
[7] A petrochemical process for producing at least either of ethylene, propylene, butene, benzene or toluene by carrying out a thermal decomposition reaction at least using naphtha as the main raw material, comprising:

hydrogenating cracked kerosene produced from a thermal cracking furnace by the method described in any of [1] to [6] above,

followed by re-supplying all or a portion of the hydrogenated hydrocarbons to the thermal cracking furnace.

[8] The petrochemical process described in [7] above, wherein the proportion of unsaturated carbon atoms in the hydrogenated hydrocarbons re-supplied to the thermal cracking furnace is 20 mol % or less based on the total number of carbon atoms in the hydrogenated hydrocarbons.
[9] The petrochemical process described in [7] or [8] above,

wherein the ratio of hydrogen to cracked kerosene supplied to hydrogenation reaction of the first stage is such that hydrogen gas/cracked kerosene=140 to 10000 Nm³/m³.

[10] The petrochemical process described in any of [7] to [9] above, wherein a portion of the hydrocarbons hydrogenated in the second stage are mixed with cracked kerosene followed by supplying this mixture to a hydrogenation reaction in the first stage.
[11] The petrochemical process described in any of [7] to [10] above, wherein the hydrogen supplied to the second stage of hydrogenation is hydrogen produced from a thermal cracking furnace.
[12] The petrochemical process described in any of [7] to [11] above, wherein all or at least a portion of the unreacted hydrogen in the hydrogenation reaction is re-supplied to the hydrogenation reaction.
[13] The petrochemical process described in [12] above, wherein all or at least a portion of hydrogen sulfide contained in the unreacted hydrogen is removed followed by re-supplying the unreacted hydrogen to the hydrogenation reaction.
[14] The petrochemical process described in any of [7] to [13] above, wherein the total sulfur concentration in the cracked kerosene supplied to the hydrogenation reaction is 1000 ppm or less by weight.

As has been described above, according to the present invention, useful components such as ethylene and propylene can be obtained at high yield without causing fouling of a thermal cracking furnace by coking. Moreover, prolongation of catalyst life is achieved since coking of the hydrogenation catalyst is prevented.

### BRIEF DESCRIPTION OF THE DRAWINGS

FIG. 1 is a schematic drawing showing a process for obtaining raw materials for petrochemical cracker by a two-stage hydrogenation reaction of cracked kerosene;

FIG. 2 is a schematic drawing showing a process as shown in FIG. 1 in which a portion of the hydrogenation reaction product liquid is re-supplied to a two-stage hydrogenation reaction;

FIG. 3 is a schematic drawing showing a process as shown in FIG. 2 in which hydrogen formed from an ethylene plant (Thermal decomposition process) is supplied to a two-stage hydrogenation reaction;

FIG. 4 is a schematic drawing showing a process as shown in FIG. 3 in which unreacted hydrogen gas is re-supplied to a two-stage hydrogenation reaction;

FIG. 5 is a schematic drawing showing a process as shown in FIG. 4 in which hydrogen sulfide in unreacted hydrogen gas is desulfurized and supplied to a two-stage hydrogenation reaction;

FIG. 6 is a block drawing showing one embodiment of a process for obtaining raw materials for petrochemical cracker from cracked kerosene; and

FIG. 7 is a block drawing showing an overview of a laboratory experimental device.

### BRIEF DESCRIPTION OF THE REFERENCE SYMBOLS

11: Ethylene production plant
12: Pump
13: 1st stage hydrogenation reactor
14: PSA (pressure swing adsorption) unit
15: Compressor
16: Compressor
17: 2nd stage hydrogenation reactor
18: Separation device
19: Pump
20: Hydrogen sulfide removal tower

### BEST MODE FOR CARRYING OUT THE INVENTION

The following provides a detailed explanation of embodiments of the present invention with reference to the drawings.

<Mixture of Hydrocarbon Compounds Having Aromatic Ring and/or Ethylenic Carbon-Carbon Double Bonds>

The “mixture of hydrocarbon compounds having aromatic ring and/or ethylenic carbon-carbon double bonds” of the present invention refers to a mixture containing at least one type or two or more types of compounds selected from the group consisting of hydrocarbon compounds having both aromatic rings, hydrocarbon compounds having ethylenic carbon-carbon double bonds, and hydrocarbon compounds having aromatic rings and ethylenic carbon-carbon double bonds. In addition, examples of these mixtures of hydrocarbon compounds include comparatively high boiling point fractions produced by thermal decomposition of naphtha in an ethylene plant, and particularly a fraction referred to as cracked kerosene or cracked heavy oil (IBP (initial boiling point): 187° C., 50% distillation temperature: 274° C.).

More specifically, hydrocarbon compounds having aromatic rings are compounds such as benzene or naphthalene. In addition, these may include aromatic heterocyclic compounds. Examples of groups having ethylenic carbon-carbon double bonds include vinyl groups, allyl groups and ethenyl groups, while typical examples of hydrocarbon compounds having such groups include olefins such as ethylene or butene. Examples of compounds having both aromatic ring and ethylenic carbon-carbon double bonds include styrene or vinyltoluene.

Furthermore, the present invention can be applied to not only cracked kerosene, but also mixtures of hydrocarbon compounds having aromatic ring and/or ethylenic carbon-carbon double bonds in general. However, in the present description, an explanation is provided of the example of cracked kerosene as the hydrogenation raw material to avoid redundancy of the notation.

Thus, in the present description, “cracked kerosene” includes the aforementioned “mixtures of hydrocarbon compounds having aromatic ring and/or ethylenic carbon-carbon double bonds in general” unless specifically indicated otherwise.

<Cracked Kerosene>

The cracked kerosene of the present invention refers to a mixture of unsaturated hydrocarbon compounds mainly having 9 or more carbon atoms produced by thermal decomposition of naphtha, and that is a fraction having a boiling point at 1 atm within the range of 90 to 230° C. However, since the cracked kerosene of the present invention is a mixture of various hydrocarbon compounds, there may be slight variations in the number of carbon atoms and boiling point.

Examples of the main components of cracked kerosene include toluene, ethylbenzene, xylene, styrene, propylbenzene, methylethylbenzene, trimethylbenzene, methylstyrene, vinyltoluene, dicyclopentadiene, indane, indene, diethylbenzene, methylpropylbenzene, methylpropenylbenzene, ethenylethylbenzene, methylphenylcyclopropane, butylbenzene, phenylbutadiene, methylindene, naphthalene, methylnaphthalene, biphenyl, ethylnaphthalene, dimethylnaphthalene, methylbiphenyl, fluorene and phenanthrene.

<Hydrogenation Reaction>

In the hydrogenation reactions of the present invention, aromatic carbon-carbon double bonds and ethylenic carbon-carbon double bonds present in a mixture of hydrocarbon compounds such as cracked kerosene having aromatic ring and/or ethylenic carbon-carbon double bonds are hydrogenated in two stages.

More specifically, the 1st stage hydrogenation reaction is carried out at a comparatively low temperature to obtain saturated hydrocarbons by hydrogenating mainly ethylenic carbon-carbon double bonds of vinyl groups and the like, while the 2nd stage hydrogenation reaction is carried out at a high temperature to hydrogenate aromatic carbon-carbon double bonds that are difficult to hydrogenate at low temperatures due to their chemical stability.

On the other hand, if the reaction temperature is raised from the start (equivalent to the case of carrying out the 2nd stage reaction first), simultaneous to the hydrogenation reaction of ethylenic carbon-carbon double bonds, polymerization reactions by ethylenic carbon-carbon double bonds also end up proceeding. The polymers accumulate on the surface of the hydrogenation catalyst causing a decrease in catalyst activity while also shortening the catalyst life. Moreover, the polymers also cause the problem of fouling in which polymers adhere to and accumulate on the inner walls of the reaction pipes.

In contrast, under the conditions of the 1st stage hydrogenation reaction according to the present invention, since it is difficult for the polymerization reaction to occur, ethylenic carbon-carbon double bonds are consumed by the hydrogenation reaction. Thus, even if the temperature is raised in the 2nd stage hydrogenation reaction, since there are hardly any ethylenic carbon-carbon bonds to polymerize, the aforementioned problems associated with catalyst poisoning do not occur.

Furthermore, this process is not limited to the aforementioned two-stage reaction, but rather is a process that at least includes the aforementioned two stages. Namely, reactions or treatment steps for achieving other objectives may be included either before or after or during the aforementioned two reaction stages.

The following specifically indicates the hydrogenation reaction conditions of each stage.

<(I) 1st Stage Hydrogenation Reaction>

Temperature: 50 to 180° C.

Pressure: 1 to 8 MPa

Time: 0.01 to 2 hours

Raw material ratio: Hydrogen gas/cracked kerosene=140 to 10000 Nm³/m³

Catalyst: Pt, Pd, etc.

The 1st stage hydrogenation reaction consists of hydrogenating mainly ethylenic carbon-carbon double bonds by contacting hydrogen gas and cracked kerosene in the presence of a hydrogenation catalyst.

The 1st stage reaction temperature is preferably 50 to 180° C. If the reaction temperature is lower than 50° C., the conversion rate of the hydrogenation reaction decreases. On the other hand, if the reaction temperature exceeds 180° C., there is the risk of the occurrence of thermal polymerization of the ethylenic carbon-carbon double bonds. Thus, the 1st stage reaction temperature is preferably 50 to 180° C., more preferably 80 to 150° C. and even more preferably 90 to 120° C.

The pressure during the 1st stage reaction is preferably 1 to 8 MPa. If the pressure during the reaction is lower than 1 MPa, the conversion rate of the hydrogenation reaction decreases. On the other hand, if the pressure during the reaction exceeds 8 MPa, there is the disadvantage of increased equipment costs. Thus, the pressure during the 1st stage reaction is preferably 1 to 8 MPa, more preferably 3 to 7 MPa and even more preferably 4 to 6 MPa.

The 1st stage reaction time is preferably 0.01 to 2 hours. If the reaction time is less than 0.01 hours, the hydrogenation conversion rate decreases. On the other hand, if the reaction time exceeds 2 hours, the amount of hydrogenation catalyst relative to the cracked kerosene to be treated becomes excessive and a large reactor is required, thereby making this economically disadvantageous. Thus, the 1st stage reaction time is preferably 0.01 to 2 hours, more preferably 0.1 to 1 hour and even more preferably 0.15 to 0.5 hours.

The ratio of hydrogen gas to cracked kerosene is preferably 140 to 10000 Nm³/m³. If the ratio of hydrogen gas to cracked kerosene is less than 140 Nm³/m³, the hydrogenation conversion rate decreases. On the other hand, if the ratio of hydrogen gas to cracked kerosene exceeds 10000 Nm³/m³, a large amount of the hydrogen gas is unconverted making this economically disadvantageous. Thus, the ratio of hydrogen gas to cracked kerosene is preferably 140 to 10000 Nm³/m³, more preferably 1000 to 8000 Nm³/m³ and even more preferably 2000 to 6000 Nm³/m³.

There are no particular limitations on the catalyst provided for the 1st stage hydrogenation reaction provided it has the ability to hydrogenate olefins. In addition, it may not have the ability to hydrogenate aromatic rings. In general, a catalyst containing a metal component such as Pt, Pd, Ni or Ru can be used. In addition, these catalysts may be supported onto a carrier. Examples of carriers include alumina, activated carbon, zeolite, silica, titania and zirconia. More specifically, a hydrogenation catalyst described in the Japanese Patent No. 3463089 can be used.

The degree of the 1st stage hydrogenation reaction can be evaluated according to the bromine number (JIS K 2605), which is an indicator of ethylenic carbon-carbon double bonds remaining without being hydrogenated. The bromine number of the product of this reaction is preferably 20 g/100 g or less. In the case the bromine number exceeds 20 g/100 g, this indicates that a large number of ethylenic carbon-carbon double bonds remain, thereby increasing the catalyst deterioration rate in the 2nd stage high-temperature hydrogenation reaction due to polymerization of these ethylenic carbon-carbon double bonds on the surface of the catalyst. Thus, the bromine number of the 1st stage hydrogenation reaction is preferably 20 g/100 g or less, more preferably 10 g/100 g or less and even more preferably 5 g/100 g or less.

<(II) 2nd Stage Hydrogenation Reaction>

Temperature: 230 to 350° C.

Pressure: 1 to 8 MPa

Time: 0.01 to 2 hours

Raw material ratio: Hydrogen gas/1st stage reaction product=140 to 10000 Nm³/m³

Catalyst: Pt, Pd, Ru, Ni, Rh, etc.

The 2nd stage hydrogenation reaction consists of hydrogenating mainly aromatic carbon-carbon double bonds by contacting hydrogen gas and the 1st stage reaction product in the presence of a hydrogenation catalyst. This reaction also promotes the hydrogenation of ethylenic carbon-carbon double bonds that did not react in the 1st stage.

The 2nd stage reaction temperature is preferably 230 to 350° C. If the reaction temperature is lower than 230° C., the aromatic carbon-carbon double bonds are not adequately hydrogenated. On the other hand, if the reaction temperature exceeds 350° C., carbon precipitates on the catalyst, hot spots are formed due to the heat of the reaction, and the reaction equilibrium shifts from hydrogenation to dehydrogenation, and these are disadvantageous for the hydrogenation reaction and catalyst life. Thus, the 2nd stage reaction temperature is preferably 230 to 350° C., more preferably 240 to 330° C. and even more preferably 260 to 300° C.

The pressure during the 2nd stage reaction is 1 to 8 MPa, preferably 3 to 7 MPa and more preferably 4 to 6 MPa. If the pressure is lower than 1 MPa, the aromatic carbon-carbon double bonds are not adequately hydrogenated, thereby making this undesirable. In particular, in the case of hydrogenation of a raw material containing sulfur compounds in the manner of cracked kerosene, it is necessary to prevent poisoning of the precious metal catalyst with a high hydrogen pressure. If the pressure exceeds 8 MPa, equipment costs, operating costs and the like increase, thereby making this undesirable.

The 2nd stage reaction time is preferably 0.01 to 2 hours. If the reaction time is less than 0.01 hours, the aromatic carbon-carbon double bonds may not be adequately hydrogenated. On the other hand, if the reaction time exceeds 2 hours, the amount of hydrogenation catalyst relative to the cracked kerosene to be treated becomes excessive and a large reactor is required, thereby making this economically disadvantageous. Thus, the 2nd stage reaction time is preferably 0.01 to 2 hours, more preferably 0.1 to 1 hour and even more preferably 0.15 to 0.5 hours.

The same hydrogen gas as that used in the 1st stage can be used for the hydrogen gas provided for the 2nd stage hydrogenation reaction. In addition, fresh hydrogen gas is not required to be supplied, but rather the hydrogenation reaction may be carried out by supplying the 1st stage reaction product and unreacted hydrogen gas to the 2nd stage reactor as is.

The ratio of hydrogen gas to the 1st stage reaction product is preferably 140 to 10000 Nm³/m³. If the ratio of hydrogen gas to the 1st stage reaction product is less than 140 Nm³/m³, the hydrogenation conversion rate decreases. In addition, if the ratio of hydrogen gas to the 1st stage reaction product exceeds 10000 Nm³/m³, a large amount of the hydrogen gas is unconverted making this economically disadvantageous. Thus, the ratio of hydrogen gas to the 1st stage reaction product is preferably 140 to 10000 Nm³/m³, more preferably 1000 to 8000 Nm³/m³ and even more preferably 2000 to 6000 Nm³/m³.

There are no particular limitations on the catalyst provided for the 2nd stage hydrogenation reaction provided it has the ability to hydrogenate an aromatic ring, and typically a catalyst containing a metal component such as Pt, Pd, Ni, Ru or Rh can be used. In addition, these catalysts may be supported onto a carrier. Examples of carriers include alumina, activated carbon, zeolite, silica, titania and zirconia. Examples of these catalysts include Ru/carbon, Ru/alumina, Ni/diatomaceous earth, Rainey nickel, supported Rh, Ru/Co/alumina and Pd/Ru/carbon. More specifically, a hydrogenation catalyst described in the Japanese Patent No. 3463089 can be used.

Since the 2nd stage catalyst for hydrogenating aromatic carbon-carbon double bonds can also be used to hydrogenate ethylenic carbon-carbon double bonds, this catalyst can also be used in the 1st stage hydrogenation reaction, and the same catalyst may be used in both the 1st stage and 2nd stage reactions.

Cracked kerosene is known to normally contain several ten to several thousand ppm of sulfur compounds. These sulfur compounds contain thiols, sulfides, thiophenes, benzothiophenes, dibenzothiophenes and the like. Although the aforementioned metal-based catalysts demonstrate high nuclear hydrogenation activity even under comparatively mild conditions and are suitable for use in both the 1st stage and 2nd stage reactions, there are cases in catalyst life may be shortened as a result of being poisoned by sulfur compounds. Thus, it is preferable to reduce the amount of sulfur compounds contained in cracked kerosene as raw materials supplied to a hydrogenation reaction. The total sulfur concentration of raw materials supplied to a hydrogenation reaction in terms of the weight ratio thereof is preferably 1000 ppm or less, more preferably 500 ppm or less and even more preferably 200 ppm or less. In cases in which the cracked kerosene has a high total sulfur concentration, it is preferable to incorporate a desulfurization device before the hydrogenation reaction step.

In addition, the problems caused by sulfur compounds as described above can be improved by supported platinum or palladium onto an ultrastabilized Y zeolite carrier having solid acidity. The use of these catalysts is also preferable in the hydrogenation reactions of the present invention. Japanese Unexamined Patent Application, First Publication No. H11-57482 discloses that resistance to sulfur poisoning is further improved in the case of hydrogenating a sulfur-containing aromatic hydrocarbon oil by using a catalyst in which a Pd—Pt precious metal species is supported onto a zeolite carrier modified with cerium (Ce), lanthanum (La), magnesium (Mg), calcium (Ca) or strontium (Sr). Moreover, U.S. Pat. No. 3,463,089 discloses that the dearomatization rate of light oil or n-hexadecane solutions of tetralin containing sulfur and nitrogen can be improved considerably by supporting platinum or palladium, and a third component in the form of ytterbium (Yb), gadolinium (Gd), terbium (Tb) or dysprosium (Dy), onto an ultrastabilized Y zeolite (USY zeolite) carrier having solid acidity.

Hydrogen gas supplied to the 1st stage and 2nd stage hydrogenation reactions may be pure hydrogen or contain low activity substances such as methane in the manner of hydrogen produced from a thermal cracking furnace using naphtha as the main raw material. In addition, in the case of a containing precious metal catalyst poisonous substance like carbon monoxide, it is desirable to purify the hydrogen gas by separating the carbon monoxide using pressure swing adsorption (PSA) or membrane separation and the like. In addition, it is also economically effective to re-pressurize and re-supply hydrogen not consumed in the reactions to the reactor after vapor-liquid separation with condensed components at the reactor outlet.

In these reactions, there are cases in which sulfur compounds present in the raw material liquid (cracked kerosene) may be converted to hydrogen sulfide by a desulfurization reaction. In such cases, there is the possibility that all or a portion of the hydrogen sulfide generated in the desulfurization reaction is contained in hydrogen re-supplied to the reactor. Since this hydrogen sulfide has the potential to promote deterioration of the catalyst used in the reaction, it is preferably removed prior to being supplied to the reactor. Typical examples of methods for removing hydrogen sulfide include removal by reacting with sodium hydroxide (chemical method) and removal by adsorption using iron (iron powder method). This removal of hydrogen sulfide may be carried out after vapor-liquid separation with condensed components or after pressurizing hydrogen gas re-supplied to the reactor.

Since the 1st stage and 2nd stage hydrogenation reactions can adopt similar reaction forms, a fixed bed adiabatic reactor or fixed bed multitubular reactor may be used for the type of reactor used in the reactions. Since hydrogenation reactions generate a large heat of reaction, a process that enables this heat of reaction to be removed is preferable. For example, in the case of using a fixed bed adiabatic reactor, the heat of reaction can be removed or hot spots can be avoided by supplying a large amount of liquid or gas for dissipating heat. In addition, in the case of using a fixed bed multitubular reactor, since heat can be removed without having to supply a large amount of liquid or gas for dissipating heat, this reactor offers the advantage of being able to reduce operating costs. It is necessary to remove the heat of reaction as described above since side reactions such as hydrogenolysis, precipitation of carbon, loss of reaction control and other undesirable phenomena occur if the temperature rise in the catalyst layer exceeds 50° C.

The form of the reaction in the reactor may be in the form of an upflow or downflow. In the case the reaction is a downflow type of gas-solid-liquid reaction, a method consisting of the installation of a liquid dispersion plate and the like inside the reactor is used to prevent flow distortion.

There are no particular limitations on the form of the catalyst, examples of catalyst forms include powders, columns, spheres, lobes and honeycombs, and the form of the catalyst can be suitably selected according to conditions of use. Among these, regularly shaped catalysts such as columnar, spherical, lobular or honeycomb-shaped catalysts are preferable in the aforementioned fixed bed reaction devices.

Normally, it is necessary to avoid the formation of hot spots in the catalyst packed layer since hydrogenation reactions are accompanied by a large heat of reaction. In general, it is necessary to, for example, dilute the supplied liquid with an inert solvent, dilute the catalyst with an inert carrier or quench the catalyst with hydrogen gas. In the case of diluting the supplied liquid with an inert solvent, it is preferable, in consideration of costs required to separate and refine the product, to recycle a portion of the reaction product of the process and mix with cracked kerosene. In addition, avoiding hot spots also prevents polymerization of vinyl groups, thereby making it possible to significantly reduce the rate of catalyst deterioration caused by coking.

The degree of the 2nd stage hydrogenation reaction can be evaluated by measuring aromatic ring and/or ethylenic carbon-carbon double bonds remaining without being hydrogenated by ¹³C-NMR. The proportion of unsaturated carbon in the 2nd stage reaction product is preferably 20% or less. In the case the proportion of unsaturated carbon in the reaction product exceeds 20%, the decomposition yield of substances containing an aromatic ring in the cracking furnace becomes extremely low, thereby preventing the obtaining of an adequate amount of high added value products even if supplied to a thermal decomposition step and the obtaining of an industrially meaningful process. Thus, the proportion of unsaturated carbon in the 2nd stage reaction product is preferably 20% or less, more preferably 10% or less and even more preferably 5% or less.

The following provides a definition of the proportion of unsaturated carbon.

(Proportion of unsaturated carbon)=(molar amount of unsaturated carbon atoms)/(molar amount of all carbon atoms contained in product following 2nd stage of hydrogenation)×100[%]

Furthermore, unsaturated carbon atoms refer to carbon atoms bound in an unsaturated manner regardless of whether or not they are conjugated. For example, the number of unsaturated carbons in the case of propylene is 2 (total number of carbon atoms: 3), while the number of unsaturated carbons in the case of toluene is 6 (total number of carbon atoms: 7).

<Process>

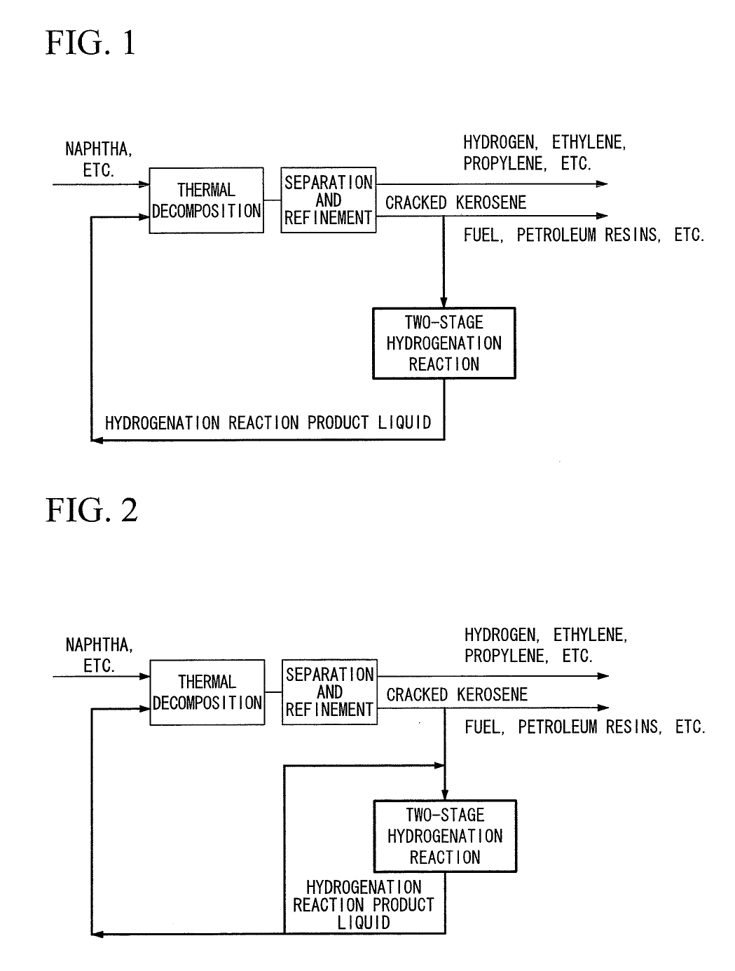

The following provides an explanation of the petrochemical process of the present invention (to simply be referred to as the “process”) with reference to FIGS. 1 to 5.

FIG. 1 shows a process for obtaining the raw materials for petrochemical cracker by a two-stage hydrogenation reaction of cracked kerosene.

In the process shown in FIG. 1, a petrochemical raw material such as naphtha is cracked in a high-temperature thermal cracking furnace followed by refining and separating the decomposition product thereof to produce hydrogen, ethylene, propylene, cracked kerosene and the like. In addition, the cracked kerosene obtained following thermal decomposition, refining and separation is ordinarily used as fuels, a raw material for petroleum resins and the like. This process hydrogenates aromatic ring and/or ethylenic carbon-carbon double bonds contained in all or a portion of the cracked kerosene by a two-stage hydrogenation reaction as previously described, and recirculates these hydrogenated hydrocarbons to a thermal cracking furnace as raw materials.

FIG. 2 shows a process for obtaining the raw materials for petrochemical cracker by re-supplying a portion of the liquid following the hydrogenation reaction to two-stage hydrogenation reaction in the process shown in FIG. 1.

In the process shown in FIG. 2, increases in catalyst layer temperature or catalyst surface temperature are inhibited by re-circulating a portion of the Hydrogenation reaction product liquid resulting from hydrogenation of aromatic ring and/or ethylenic carbon-carbon double bonds obtained in the process shown in FIG. 1 to a two-stage hydrogenation reaction. As a result, adhesion of coke to the catalyst surface decreases thereby enabling considerable improvement of catalyst life.

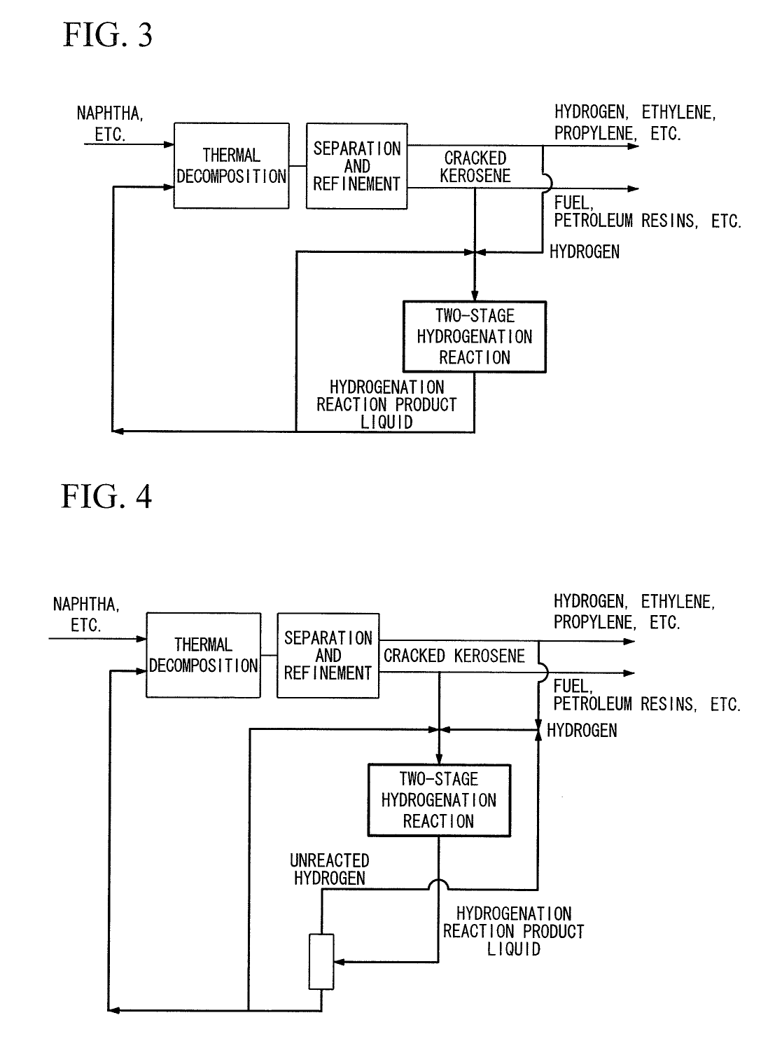

FIG. 3 shows a process for obtaining raw materials for petrochemical cracker by further supplying hydrogen produced from an ethylene plant to a two-stage hydrogenation reaction in the process shown in FIG. 2.

In the process shown in FIG. 3, hydrogen produced from an ethylene plant is supplied to a two-stage hydrogenation reaction. There are no restrictions on the generation source of the hydrogen supplied to the hydrogenation reaction. The hydrogen may be one produced from a thermal cracking furnace. Impurities such as methane or carbon monoxide can be removed by a method such as PSA as necessary.

FIG. 4 shows a process by further re-supplying unreacted hydrogen gas to a two-stage hydrogenation reaction in the process shown in FIG. 3.

In the process shown in FIG. 4, unreacted hydrogen among the hydrogen supplied to the two-stage hydrogenation reaction is re-supplied to a two-stage hydrogenation reaction. Hydrogen supplied to the two-stage hydrogenation reaction is normally supplied in excess relative to the required theoretical amount in order to hydrogenate aromatic ring and/or ethylenic carbon-carbon double bonds present in the cracked kerosene. Consequently, unreacted hydrogen is present at the reactor outlet, and the reuse of this hydrogen in a hydrogenation reaction results in even greater efficiency in terms of economy.

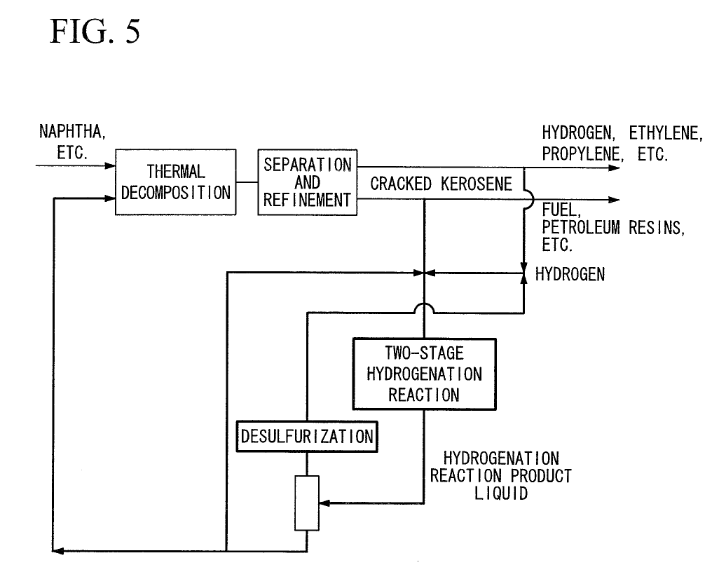

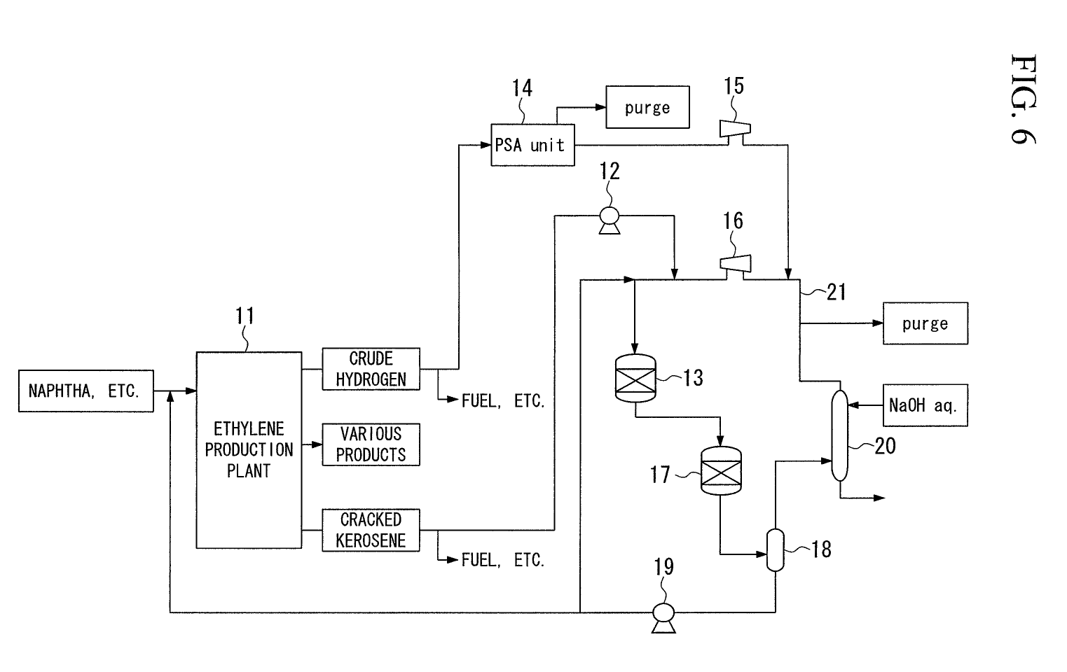

FIG. 5 shows a process by desulfurizing hydrogen sulfide present in unreacted hydrogen gas before re-supplying the hydrogen gas to a two-stage hydrogenation reaction in the process shown in FIG. 4.

In the process shown in FIG. 5, the unreacted hydrogen is re-supplied to the hydrogenation reaction after having removed hydrogen sulfide contained therein. In addition, in this process, hydrogen sulfide present in the unreacted hydrogen is also removed to avoid concentration of hydrogen sulfide in the hydrogen circulation system. Cracked kerosene normally contains sulfur compounds, and all or a portion of these sulfur compounds react in the two-stage hydrogenation reaction to form hydrogen sulfide. Hydrogen sulfide has a low boiling point, and is contained in unreacted hydrogen when the unreacted hydrogen is re-circulated. In addition, this hydrogen sulfide may also be a catalyst poison of the hydrogenation catalyst. Thus, in this process, this problem can be avoided by removing the hydrogen sulfide.

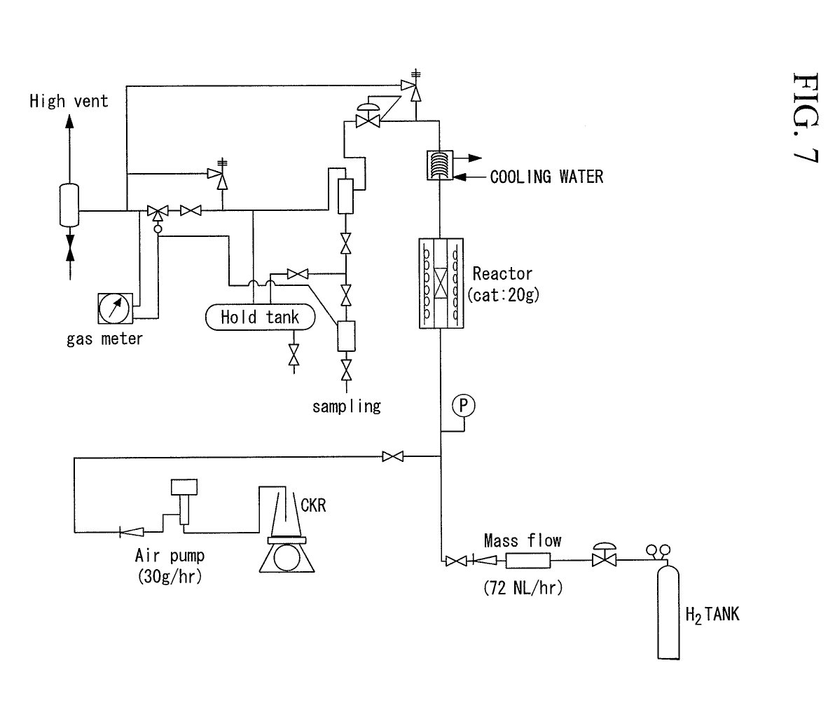

Although the above has provided a general explanation of the process of the present invention, the following provides a more detailed explanation of an embodiment of the process with reference to FIG. 6.

As shown in FIG. 6, in this process, a petrochemical raw material such as naphtha is thermally decomposed and refined in ethylene plant 11 to produce various products such as ethylene and propylene. All or a portion of the cracked kerosene among this group of products is pressurized by a pump 12 and supplied to a 1st stage hydrogenation reactor 13. On the other hand, the hydrogen concentration of a mixed gas of hydrogen, methane and carbon monoxide obtained from the ethylene plant 11 is increased with a PSA unit 14 followed by pressurizing this hydrogen-rich gas with a compressor 15. After mixing this hydrogen-rich gas with circulating hydrogen gas 21, the pressure is further increased by a compressor 16 followed by supplying to the 1st stage hydrogenation reactor 13. In the 1st stage hydrogenation reactor 13, hydrogen gas and cracked kerosene are contacted in the presence of a hydrogenation catalyst to mainly carry out hydrogenation of ethylenic carbon-carbon double bonds. Gas and the like discharged from the 1st stage hydrogenation reactor 13 is supplied to a 2nd stage hydrogenation reactor 17. In the 2nd stage hydrogenation reactor 17, hydrogen gas and the 1st stage reaction product are contacted in the presence of a hydrogenation catalyst to mainly carry out hydrogenation of aromatic carbon-carbon double bonds. As a result, hydrogenation of ethylenic carbon-carbon double bonds that did not react in the 1st stage also proceeds. Gas and the like discharged from the 2nd stage hydrogenation reactor 17, namely unreacted hydrogen gas containing hydrogen sulfide and a reaction liquid subjected to hydrogenation treatment of aromatic ring and/or ethylenic carbon-carbon double bonds by the aforementioned two-stage hydrogenation reaction, is subjected to gas-liquid separation by a separation device 18 provided at the outlet of the 2nd stage hydrogenation reactor 17. A portion of a condensed liquid thereof is pressurized by a pump 19 and re-circulated to the 1st stage hydrogenation reactor 13. In addition, a portion of the condensed liquid is re-supplied to the thermal cracking furnace of the ethylene plant 11 as raw materials for cracker. On the other hand, non-condensing gas consisting mainly of unreacted hydrogen gas containing hydrogen sulfide is subjected to washing treatment with an aqueous sodium hydroxide solution in a hydrogen sulfide removal tower 20, followed by mixing with fresh hydrogen gas from the compressor 15. After being pressurized by the compressor 16, the mixture is supplied to the 1st stage hydrogenation reactor 13. Furthermore, in this process, all or a portion of the unreacted hydrogen gas may be purged outside the system.

<Decomposition Reaction Simulation>

In the case of reusing a hydrogenation product obtained from a process as described above, in which aromatic ring and/or ethylenic carbon-carbon double bonds have been reduced, as a raw material of thermal cracking furnace, the thermal decomposition yield of ethylene, propylene and the like is extremely high as compared with the case of using cracked kerosene as is for a raw material of thermal cracking furnace.

Here, a decomposition reaction simulation was carried out for the components of samples (1) to (4) below, and results based on the presumed compositions of the products are shown in Table 2.

(1) Cracked kerosene
(2) Cracked kerosene in which all unsaturated carbons, including aromatic ring carbon-carbon double bonds, have been hydrogenated, and the proportion of unsaturated carbon is presumed to be 0% of all carbon present
(3) Cracked kerosene in which unsaturated carbons other than aromatic ring carbon-carbon double bonds are presumed to have been hydrogenated

### (4) Naphtha

Furthermore, thermal decomposition yield was calculated using the process simulator described below.

Calculation software: SPYRO Ethylene Decomposition Tube Decomposition Yield Calculation Software, Technip Ltd.

Decomposition temperature: 818° C.

Steam/raw material hydrocarbon ratio: 0.4/1.0 (wt/wt)

In addition, the supply compositions of samples (1) to (4) were as indicated below.
(1) Cracked kerosene:

Cyclopentadiene (0.5% by weight), methylcyclopentadiene (2.0% by weight), benzene (0.5% by weight), toluene (1.0% by weight), ethylbenzene (7.0% by weight), styrene (9.0% by weight), dicyclopentadiene (5.0% by weight), vinyltoluene (25% by weight), indene (22% by weight), naphthalene (4.0% by weight), 1,3,5-trimethylbenzene (4.0% by weight), 1,2,4-trimethylbenzene (6.0% by weight), 1,2,3-trimethylbenzene (4.0% by weight), α-methylstyrene (3.0% by weight), β-methylstyrene (4.0% by weight), methylindene (3.0% by weight) (initial boiling point: 101.5° C., endpoint: 208.5° C., density: 0.92 g/L, bromine number: 100 g/100 g)
(2) Cracked kerosene in which all unsaturated carbons have been hydrogenated:

Cyclopentane (0.5% by weight), methylcyclopentane (2.0% by weight), cyclohexane (0.5% by weight), methylcyclohexane (1.0% by weight), ethylcyclohexane (16% by weight), dicyclopentane (5.0% by weight), 1-methyl-4-ethylcyclohexane (25% by weight), hydrindane (22% by weight), decalin (4.0% by weight), trimethylcyclohexane (14% by weight), isopropylcyclohexane (3.0% by weight), n-propylcyclohexane (4.0% by weight), methylhydrindan (3.0% by weight)
(3) Cracked kerosene in which unsaturated carbons other than aromatic ring carbon-carbon double bonds have been hydrogenated:

Cyclopentadiene (0.5% by weight), methylcyclopentadiene (2.0% by weight), benzene (0.5% by weight), toluene (1.0% by weight), ethylbenzene (16% by weight), dicyclopentadiene (5.0% by weight), methylethylbenzene (25% by weight), indane (22% by weight), naphthalene (4.0% by weight), 1,3,5-trimethylbenzene (4.0% by weight), 1,2,4-trimethylbenzene (6.0% by weight), 1,2,3-trimethylbenzene (4.0% by weight), n-propylbenzene (3.0% by weight), cumene (4.0% by weight), methylindane (3.0% by weight)
(4) Naphtha:

Normal paraffin components: C3 (0.03% by weight), C4 (2.2% by weight), C5 (9.8% by weight), C6 (4.5% by weight), C7 (7.6% by weight), C8 (5.5% by weight), C9 (3.4% by weight), C10 (0.74% by weight), C11 (0.02% by weight); isoparaffin components: C4 (0.33% by weight), C5 (6.7% by weight), C6 (8.2% by weight), C7 (6.6% by weight), C8 (8.5% by weight), C9 (3.8% by weight), C10 (2.1% by weight), C11 (0.09% by weight); olefin components: C9 (0.16% by weight), C10 (0.01% by weight); naphthene components: C5 (1.2% by weight), methyl-C5 (2.5% by weight), C6 (1.2% by weight), C7 (4.3% by weight), C8 (4.2% by weight), C9 (2.8% by weight), C10 (0.47% by weight); aromatic components: benzene (0.52% by weight), toluene (1.8% by weight), xylene (2.9% by weight), ethylbenzene (0.86% by weight), C9 (2.0% by weight), C10 (0.02% by weight)

TABLE 2

| | | Sample | | | |
|---|---|---|---|---|---|
| | | (1) (wt %) | (2) (wt %) | (3) (wt %) | (4) (wt %) |
| Decompo- | Hydrogen | 0.55 | 0.55 | 0.55 | 0.55 |
| sition | Hydrogen/ | 7.1 | 15.3 | 10.0 | 15.3 |
| Products | methane | | | | |
| | Ethylene | 2.5 | 17.9 | 4.7 | 26.4 |
| | Propylene | 0.4 | 10.8 | 0.5 | 15.7 |
| | C4/C5 | 0.1 | 13.2 | 1.2 | 13.9 |
| | Cracked | 32.6 | 28.0 | 35.8 | 16.2 |
| | gasoline | | | | |
| | C9 or | 56.2 | 9.7 | 47.0 | 6.4 |
| | larger | | | | |
| | Other | 0.6 | 4.6 | 0.3 | 5.6 |

Based on the calculation results of Table 2, the thermal decomposition yields of high added value components such as ethylene and propylene useful for the petrochemical industry can be determined to be improved considerably as a result of making the proportion of unsaturated carbon of hydrocarbons 0% of all carbon present by hydrogenating aromatic ring and/or ethylenic carbon-carbon double bonds. For example, in contrast to ethylene yield being 2.5% in the case of thermal decomposition of cracked kerosene (1), the ethylene yield of cracked kerosene, in which the proportion of unsaturated carbon among all carbon present was presumed to be 0% by hydrogenating unsaturated carbon, including aromatic rings, was 17.9%. Similarly, the yield of propylene in the case of (1) was 0.4%, while that in the case of (2) was 10.8%.

### EXAMPLES

The effects of the present invention will be made clearer from the following examples. Furthermore, the present invention is not limited to the following examples, and can be carried out by suitably modifying within a scope that does not alter the gist thereof.

<Experimental Apparatus>

In the examples, a high-pressure fixed-bed flow reactor employing a configuration like that shown in FIG. 7 was used, a catalyst was packed inside the reaction tube, and hydrogenation reaction was carried out in an upflow mode. Furthermore, the 1st and 2nd stage hydrogenation reactions in Examples 1 and 2 to be described later were carried out independently, and the entire amount of the 1st stage reaction condensate was used for the raw material liquid supplied to the 2nd stage reaction.

An upright tube reactor having an inner diameter of 19.4 mm and catalyst packed effective length of 520 mm was used for the reactor, a sheath (outer diameter: 6 mm, made of SUS316) for inserting a thermocouple was installed in the center of a catalyst layer, and the temperature of the catalyst layer was measured with a thermocouple inserted therein. 1/8B SUS316 stainless steel balls were packed into the lower 200 mm of the reaction tube to serve as a preheating layer. The temperature of the reactor was adjusted with an electric furnace, and the reaction products were cooled with a heat exchanger using water for the coolant followed by reducing to nearly atmospheric pressure with a pressure control valve, separating into a condensed component and non-condensing component with a gas-liquid separator, and carrying out respective analyses on the each component. The hydrogen flow rate was controlled with a flow rate control valve. An air pump was used to supply the raw material liquid, and the supply rate was taken to be the weight reduction rate of an electronic balance on which a raw material container was placed.

<Analysis of Condensed Component (Post-Reaction Liquid Component)>

“Bromine number” was determined using the apparatus and under the conditions described below.

Apparatus: Karl Fischer Bromine Number Measuring System (MKC-210, Kyoto Electronics Manufacturing Co., Ltd.)

Counter electrode solution: 0.5 mol/L aqueous potassium chloride solution, 5 mL

Electrolyte: 1 mol/L aqueous potassium bromide solution: 14 mL+guaranteed reagent grade glacial acetic acid: 60 mL+methanol: 26 mL

Sample: 10 μL injected with a microsyringe

C=(TS−TB)×F/(D×V×10⁶)×100

C: bromine number (g/100 g), TS: titrated amount (μg), TB: blank (μg), F: conversion coefficient (8.878) (no units), D: density (g/mL), V: sample volume (mL)

“Proportion of aromatic and/or ethylenic carbon-carbon double bonds” was determined using the apparatus and under the conditions described below.

Apparatus: ¹³C-NMR, 400 MHz (EX-400, JEOL Ltd.)

Measurement method: Dissolved in deuterated chloroform, tetramethylsilane used for internal standard material

“Total sulfur concentration” was determined using the apparatus and under the conditions described below.

Apparatus: Chlorine/sulfur analyzer (Model TSX-10, Mitsubishi Kasei Corp.)

Electrolyte: 25 mg sodium azide aqueous solution: 50 mL+glacial acetic acid: 0.3 mL+potassium iodide: 0.24 g

Dehydration liquid: Phosphoric acid: 7.5 mL+pure water: 1.5 mL

Counter electrode solution: 10% by weight aqueous guaranteed reagent grade potassium nitrate solution

Oxygen supply pressure: 0.4 MpaG

Argon supply pressure: 0.4 MpaG

Sample inlet temperature: 850 to 950° C.

Sample: 30 μL injected with a microsyringe

<Analysis of Non-Condensing Component (Post-Reaction Gas Component)>

“Hydrogen sulfide” was analyzed under the following conditions using the absolute calibration curve method by sampling 50 mL of effluent gas, and allowing the entire amount to flow into a 1 mL gas sampler provided with a gas chromatography system.

Apparatus: Gas chromatograph (GC-2104, Shimadzu Mfg. Co., Ltd.) equipped with Shimadzu Gas Chromatograph Gas Sampler (MGS-4, measuring tube: 1 mL)

Column: TC-1 capillary column (length: 60 m, inner diameter: 0.25 μm, film thickness: 0.25 μm)

Carrier gas: helium (flow rate: 33.5 ml/min, split ratio: 20)

Temperature conditions: detector: 300° C., vaporizing chamber: 300° C., column: constant at 80° C.

Detector: FPD (H₂ pressure: 105 kPaG, air pressure: 35 kPaG)

The “hydrogenation catalyst” was prepared in accordance with Example 2 in “Japanese Patent No. 3463089”. However, the supported amounts of precious metals were made to be 5.0% by weight of Yb, 0.82% by weight of Pd and 0.38% by weight of Pt. Namely, ytterbium acetate (Yb (CH₃COO)₃.4H₂O) was supported onto ultrastabilized Y zeolite (Tosoh Corp., HSZ-360HUA, SiO₂/Al₂O₃ molar ratio=13.9, H zeolite) using an impregnation method followed by drying overnight at 110° C. Next, a Pd precursor in the form of Pd[NH₃]₄Cl₂ and a Pt precursor in the form of Pt[NH₃]₄Cl₂ were respectively supported onto the Yb-impregnated supported zeolite. Subsequently, after drying for 6 hours at a temperature of 110° C. in vacuum, the catalyst was temporarily formed into a disc and then crushed followed by grading to a particle size of 22/48 mesh. The resulting catalyst was heated from normal temperature to 300° C. at a heating rate of 0.5° C./min in the presence of flowing oxygen, followed by calcining for 3 hours at 300° C. Final treatment in the form of hydrogen reduction of the catalyst was carried out in-situ during pretreatment for evaluation of activity.

### Example 1

### Hydrogenation Reaction

Cracked kerosene sampled with an ethylene plant and comprised of the following components was supplied to a hydrogenation reaction. The main properties of the supplied liquid are indicated below.

Initial boiling point: 101.5° C., endpoint: 208.5° C. (normal pressure)

Density: 0.92 g/L

Bromine number: 100 g/100 g

Sulfur content: 120 ppm by weight

Composition of main components: vinyltoluene: 19.4% by weight, indene: 16.0% by weight, dicyclopentadiene: 7.0% by weight, trimethylbenzene: 5.5% by weight, styrene: 5.2% by weight, α-methylstyrene: 3.1% by weight, β-methylstyrene: 5.1% by weight, methylindene: 1.0% by weight, naphthalene: 2.7% by weight

Reaction conditions for (I) 1st stage hydrogenation reaction:

Hydrogen pressure: 5.0 MPa, reaction temperature: 90 to 110° C., raw material supply rate: 30 g/h, hydrogen flow rate: 72 NL/h, amount of catalyst: 20 g, spatial velocity (WHSV): 1.5/h

Reaction conditions for (II) 2nd stage hydrogenation reaction:

Hydrogen pressure: 5.0 MPa, reaction temperature: 280 to 300° C., raw material supply rate: 30 g/h, hydrogen flow rate: 72 NL/h, amount of catalyst: 20 g, spatial velocity (WHSV): 1.5/h

In the reaction of (I), a calcined catalyst sample was packed into the reaction tube followed by subjecting to reduction treatment for 3 hours at 300° C. (heating rate: 1.0° C./min) in the presence of flowing hydrogen (normal pressure, 50 NL/h). Subsequently, the temperature of the catalyst layer was lowered to 100° C., and after pressurizing to a prescribed hydrogen pressure, raw material was introduced into a preheated portion. In addition, in the reaction of (II), the temperature of the catalyst layer was lowered to 280° C. following a similar reduction treatment, and after pressurizing to a prescribed hydrogen pressure, the reaction product liquid of reaction (I) (condensed component) was introduced directly into a preheated portion.

The results obtained following the reaction of (I) according to Example 1 are shown in Table 3 below, while the results for the reaction of (II) are shown in Table 4. Furthermore, the reaction product liquids shown in Tables 3 and 4 refer to the condensed components following the respective reactions, while the reaction product gas refers to the gas component obtained following the reactions.

TABLE 3

| | | Total sulfur | Hydrogen sul- | Proportion of un- |
|---|---|---|---|---|
| | Bromine number | concentration | fide concentra- | saturated carbon, |
| | of reaction | in reaction | tion in reaction | including aromatic |
| Operating | product liquid | product liquid | product gas | rings, in reaction |
| time (h) | (g/100 g) | (ppm by weight) | (ppm by volume) | product liquid (%) |
| Supplied | 100 | 120 | — | 69 |
| liquid | | | | |
| 100 | 11 | 118 | 0 | 55 |
| 200 | 11 | 117 | 0 | 54 |
| 300 | 12 | 122 | 0 | 54 |
| 400 | 15 | 115 | 0 | 55 |
| 500 | 15 | 123 | 0 | 55 |

TABLE 4

| | | Total sulfur | Hydrogen sul- | Proportion of un- |
|---|---|---|---|---|
| | Bromine number | concentration | fide concentra- | saturated carbon, |
| | of reaction | in reaction | tion in reaction | including aromatic |
| Operating | product liquid | product liquid | product gas | rings, in reaction |
| time (h) | (g/100 g) | (ppm by weight) | (ppm by volume) | product liquid (%) |
| 100 | 0 | 0 | 40 | 0 |
| 200 | 0 | 0 | 39 | 0 |
| 300 | 0 | 0 | 36 | 0 |
| 400 | 0 | 0 | 42 | 2 |
| 500 | 0 | 0 | 37 | 10 |

As shown in Tables 3 and 4, the proportion of unsaturated carbon, including aromatic rings, in the 2nd stage reaction product liquid increased to 10% after reacting for 500 hours. The cause of catalyst deterioration was presumed to be coking of the catalyst.

### Example 2

### Hydrogenation Reaction

The reaction of Example 2 was carried out in the same manner as Example 1. However, a mixture of cracked kerosene and the reaction product liquid of reaction (II) at a ratio of 1:4 (weight ratio) was used for the raw material of reaction (I). The reaction product liquid of reaction (I) (condensed component) was used as is for the raw material of reaction (II). Namely, both reaction (I) and reaction (II) were carried out in the same manner as Example 1 with the exception of making the raw material supply rate 150 g/h (of which that for the reaction product liquid of reaction (II) in Example 1 used as a diluent was 120 g/h), and making the spatial velocity 7.5/h. Furthermore, the 2nd stage reaction product liquid obtained in Example 1 was used for the diluent during initial operation (0 to 24 hours of operating time). The reaction product liquid generated in this Example 2 was used for the diluent thereafter.

The results obtained following the reaction of (I) according to Example 2 are shown in Table 5 below, while the results for the reaction of (II) are shown in Table 6.

TABLE 5

| | | Total sulfur | Hydrogen sul- | Proportion of un- |
|---|---|---|---|---|
| | Bromine number | concentration | fide concentra- | saturated carbon, |
| | of reaction | in reaction | tion in reaction | including aromatic |
| Operating | product liquid | product liquid | product gas | rings, in reaction |
| time (h) | (g/100 g) | (ppm by weight) | (ppm by volume) | product liquid (%) |
| Undiluted | 100 | 120 | — | 69 |
| supplied | | | | |
| liquid | | | | |
| 100 | 2 | 24 | 0 | 8 |
| 200 | 2 | 22 | 0 | 9 |
| 300 | 2 | 27 | 0 | 9 |
| 400 | 3 | 21 | 0 | 9 |
| 500 | 3 | 25 | 0 | 9 |
| 600 | 3 | 23 | 0 | 9 |
| 700 | 3 | 21 | 0 | 9 |
| 800 | 3 | 27 | 0 | 10 |
| 900 | 3 | 22 | 0 | 10 |
| 1000 | 3 | 24 | 0 | 10 |

TABLE 6

| | | Total sulfur | Hydrogen sul- | Proportion of un- |
|---|---|---|---|---|
| | Bromine number | concentration | fide concentra- | saturated carbon, |
| | of reaction | in reaction | tion in reaction | including aromatic |
| Operating | product liquid | product liquid | product gas | rings, in reaction |
| time (h) | (g/100 g) | (ppm by weight) | (ppm by volume) | product liquid (%) |
| 100 | 0 | 0 | 41 | 0 |
| 200 | 0 | 0 | 45 | 0 |
| 300 | 0 | 0 | 38 | 0 |
| 400 | 0 | 0 | 42 | 0 |
| 500 | 0 | 0 | 40 | 0 |
| 600 | 0 | 0 | 37 | 0 |
| 700 | 0 | 0 | 41 | 0 |
| 800 | 0 | 0 | 40 | 0 |
| 900 | 0 | 0 | 39 | 0 |
| 1000 | 0 | 0 | 37 | 0 |

As shown in Tables 5 and 6, the proportion of unsaturated carbon, including aromatic rings, in the 2nd stage reaction product liquid was maintained at 0% even after reacting for 1000 hours.

### Comparative Example 1

### Hydrogenation Reaction

The hydrogenation reaction described in Example 1 was carried out in a single step in Comparative Example 1. The reaction conditions consisted of hydrogen pressure of 5.0 MPa, reaction temperature of 280° C., raw material supply rate of 30 g/h, hydrogen flow rate of 72 NL/h, amount of catalyst of 20 g, and spatial velocity (WHSV) of 1.5/h. A calcined catalyst was packed into the reaction tube followed by heating from normal temperature to 300° C. at a heating rate of 1.0° C./min in the presence of flowing hydrogen (normal pressure, 50 NL/h), and subjecting to reduction treatment for 3 hours at 300° C. Subsequently, the temperature of the catalyst layer was lowered to 280° C. and then pressurized to a prescribed hydrogen pressure followed by introducing the raw material into a preheated portion.

The results obtained following the reaction according to Comparative Example 1 are shown in Table 7.

TABLE 7

| | | Total sulfur | Hydrogen sul- | Proportion of un- |
|---|---|---|---|---|
| | Bromine number | concentration | fide concentra- | saturated carbon, |
| | of reaction | in reaction | tion in reaction | including aromatic |
| Operating | product liquid | product liquid | product gas | rings, in reaction |
| time (h) | (g/100 g) | (ppm by weight) | (ppm by volume) | product liquid (%) |
| Supplied | 100 | 120 | — | 69 |
| liquid | | | | |
| 1 | 0 | 0 | — | 1 |
| 10 | 0 | 0 | — | 2 |
| 20 | 0 | 0 | 39 | 7 |
| 30 | 0 | 0 | 42 | 24 |
| 40 | 0 | 0 | 41 | 25 |
| 50 | 0 | 0 | 37 | 26 |
| 60 | 0 | 0 | 40 | 31 |
| 70 | 0 | 0 | 43 | 32 |

As shown in Table 7, the proportion of unsaturated carbons, including aromatic rings, was already detected at 1% in the reaction product liquid 1 hour after the start of the reaction, and that value increased to 32% after 70 hours. This is believed to have been caused by coking onto the catalyst. In addition, the time until the catalyst deteriorated was extremely short as compared with the hydrogenation method of the present invention.

### INDUSTRIAL APPLICABILITY

According to the present invention, useful components such as ethylene and propylene can be obtained at high yield without causing fouling of a thermal cracking furnace by coking. Moreover, prolongation of catalyst life is achieved since coking on the hydrogenation catalyst is prevented.

1. A hydrogenation method comprising:
hydrogenating a mixture of hydrocarbon compounds having aromatic ring and/or ethylenic carbon-carbon double bonds in the following two stages (I) and (II):
(I) carrying out a hydrogenation reaction within the range of 50 to 180° C.; and
(II) carrying out a hydrogenation reaction within the range of 230 to 350° C.

2. The hydrogenation method according to claim 1, wherein the mixture of hydrocarbon compounds having aromatic ring and/or ethylenic double bonds is a fraction consisting of hydrocarbons produced from a thermal cracking furnace using naphtha as the main raw material and having a boiling point within the range of 90 to 230° C.

3. The hydrogenation method according to claim 1, wherein a catalyst is used in the hydrogenation reaction, and the catalyst contains at least one type or two or more types of elements selected from the group consisting of palladium (Pd), platinum (Pt), ruthenium (Ru) and rhodium (Rh).

4. The hydrogenation method according to claim 3, wherein the catalyst supplied to the hydrogenation reaction further contains at least one type or two or more types of elements selected from the group consisting of cerium (Ce), lanthanum (La), magnesium (Mg), calcium (Ca), strontium (Sr), ytterbium (Yb), gadolinium (Gd), terbium (Tb), dysprosium (Dy) and yttrium (Y).

5. The hydrogenation method according to claim 3, wherein the catalyst supplied to the hydrogenation reaction is a catalyst supported onto zeolite.

6. The hydrogenation method according to claim 5, wherein the zeolite is USY zeolite.

7. A petrochemical process for producing at least either of ethylene, propylene, butene, benzene or toluene by carrying out a thermal decomposition reaction at least using naphtha as the main raw material, comprising:
hydrogenating cracked kerosene produced from a thermal cracking furnace by the method described in claim 1,
followed by re-supplying all or a portion of the hydrogenated hydrocarbons to the thermal cracking furnace.

8. The petrochemical process according to claim 7, wherein the proportion of unsaturated carbon atoms in the hydrogenated hydrocarbons re-supplied to the thermal cracking furnace is 20 mol % or less based on the total number of carbon atoms in the hydrogenated hydrocarbons.

9. The petrochemical process according to claim 7, wherein the ratio of hydrogen to cracked kerosene supplied to the hydrogenation reaction of the first stage is such that hydrogen gas/cracked kerosene=140 to 10000 Nm³/m³.

10. The petrochemical process according to claim 7, wherein a portion of the hydrocarbons hydrogenated in the second stage are mixed with cracked kerosene followed by supplying this mixture to a hydrogenation reaction in the first stage.

11. The petrochemical process according to claim 7, wherein the hydrogen supplied to the hydrogenation is hydrogen produced from a thermal cracking furnace.

12. The petrochemical process according to claim 7, wherein all or at least a portion of the unreacted hydrogen in the hydrogenation reaction is re-supplied to the hydrogenation reaction.

13. The petrochemical process according to claim 12, wherein all or at least a portion of hydrogen sulfide contained in the unreacted hydrogen is removed followed by re-supplying the unreacted hydrogen to the hydrogenation reaction.

14. The petrochemical process according to claim 7, wherein the total sulfur concentration in the cracked kerosene supplied to the hydrogenation reaction is 1000 ppm or less by weight.
